(19) United States
(12) Patent Application Publication
(10) Pub. No.: US 2005/0187516 A1
(43) Pub. Date: Aug. 25, 2005

(54) Intradermal and subcutaneous injection device

(76) Inventor: Arnold Neracher, Anieres (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/033,027

(22) Filed: Jan. 11, 2005

Related U.S. Application Data
(63) Continuation of application No. PCT/IB03/00455, filed on Jul. 8, 2003.

(30) Foreign Application Priority Data
Jul. 11, 2002 (WO) PCT/IB02/02703
Oct. 28, 2002 (WO) PCT/IB02/04494

(51) Int. Cl. A61M 5/30

(52) U.S. Cl. 604/68

(57) ABSTRACT

An injection device including a nozzle portion with an orifice, an ampoule containing a liquid to be injected, a piston, a retainer, and a force generator for generating at least two forces, including a movable partition slidably on the piston, a partition fixed to the piston, and a spring element between the moveable and fixed partitions, one of the least two forces sufficient to propel the liquid through the orifice in a jet of liquid having sufficient speed to pass through a patient's skin, and the other of the at least two forces sufficient to inject the liquid to a selected depth under the skin.

### CROSS-REFERENCE TO RELATED APPLICATION(S)

This application is a Continuation of International Patent Application PCT/CH2003/000455, filed on Jul. 8, 2003, which claims priority to International Application No. PCT/IB02/02703, filed on Jul. 11, 2002 and International Application No. PCT/IB02/04494, filed on Oct. 28, 2002, the contents of which are incorporated in their entirety by reference herein.

### BACKGROUND

The present invention relates to devices and methods for delivering, administering, injecting and dispensing substances, including hypodermic injection devices and methods for administering medications or other pharmaceutical compositions such as growth hormones, insulin, heparin and various ADN in the form of a liquid. In one embodiment, the invention relates to an injection device without a needle.

Injection devices wherein the operating or motive force is supplied by an entropic spring are known, for example, from PCT/IB02/02703 and PCT/IB02/04494. In the this type of device, the spring has an associated force/displacement curve, which depends on the type of elastomer used and its structure. The curve reflects the non-linear force/displacement characteristics of the material utilized. While well-suited for their purpose, the devices can not be optimally adapted to exhibit the ideal force/displacement profile necessary or desired for certain types or cases of injections.

### SUMMARY

An object of the present invention is to extend the range of use of injection devices by modifying the profile of the force/displacement curve associated with the operation of such devices. In accordance with the present invention, it is possible to shift the point of inflection, increase the relation between a primary force and a secondary force, and modify the slope of the primary force curve.

In one embodiment, the present invention comprises an injection device including a nozzle portion with an orifice, an ampoule containing a liquid to be injected, a piston, a retainer, and a force generator for generating at least two forces, including a movable partition slidably on the piston, a partition fixed to the piston, and a spring element between the moveable and fixed partitions, one of the least two forces sufficient to propel the liquid through the orifice in a jet of liquid having a sufficient speed to pass through a patient's skin, and the other of the at least two forces sufficient to inject the liquid to a selected depth under the skin.

In one embodiment, the present invention comprises a single or multiple use hypodermic injection device for intradermal or subcutaneous administration, without a needle, of a liquid product to be injected such as a medication or other pharmaceutical compositions, wherein the device includes an ampoule containing said liquid to be injected and at least two force generators assembled in cascade, including one or several free movable partitions sliding on the stem of a piston; a movable partition fixed to the piston; spring elements placed between the movable and the fixed partitions and acting as springs; a nozzle part having an orifice and retention means permitting maintaining, before utilization, the spring elements at a force sufficient to create in the ampoule a pressure to propel the liquid product to be injected, and during utilization, to propel the liquid product through the orifice in a manner to create a jet of liquid having a sufficient speed to pass through the patient's skin and to inject the liquid to the correct depth.

In view of the background mentioned above, an object of the present invention is to provide, in one embodiment, a needle-less or needle-free hypodermic injection device permitting extending the range of utilization of the injector by modifying the profile of the force/displacement curve associated with such devices. In the exemplary embodiments, it is possible to shift the point of inflection, increase the relation between the primary force and the secondary force, and modify the slope of the primary force curve.

In some embodiments, the present invention permits modifying the characteristics of a force/displacement curve by using one or several movable partitions between which are placed reticulated polymer disks. In some embodiments, the disks are perforated in the center and can have selected, suitable characteristics (hardness, cell, diameter, thickness, geometric form, etc.) permitting modifying the characteristics of the force/displacement curve given by a sole entropic spring.

In some embodiments, each one of the movable partitions can be equipped with or associated with a spacer permitting limiting the crushing force of the elastic block (which also may be referred to as a spring, entropic generator or the like). However, it should be appreciated that the use of spacers may not be required.

In accordance with the present invention, it is possible to obtain an inflection point at a precise value and a more abrupt slope that permits a more rapid transition between primary and secondary injection pressures, thereby limiting damage in tissues due to the force of the initial jet.

In some embodiments, the free, movable partitions are assembled in cascade or series, with or without spacers, depending upon the profile of the desired force/displacement curve, but it is also possible to combine the elastic block or blocks (or entropic spring) with a classical metal spring, a molecular spring (polysiloxane), or a rubber balloon in the form of a torus filled with nitrogen. Generally, in some embodiments, an injection device in accordance with the present invention will exhibit or provide motive or operating forces that occur in casade, series or sequentially.

For example, a rubber balloon is pressurized with nitrogen, which generates the secondary force (the low-pressure force) necessary for transferring the liquid into the body. An elastomer block flattened between the partitions generates the high pressure. Other combinations and resultant forces are within the scope of the present invention.

Another advantage of the present invention is that segmentation in cascade of the free movable partitions allows creating a doubling or tripling of different injection pressures by the use of materials displaying different force/displacement characteristics. Examples of such materials include, but are not limited to: entropic spring/metal spring; entropic spring/molecular spring; entropic spring/gas balloon; etc. In accordance with the present invention, this advantage is accomplished without having recourse to a non-linear curve as in the case of an entropic spring by itself.

Other advantages, objects and aspects of the invention will be understood with reference to the accompanying description, drawings and claims.

### BRIEF DESCRIPTION OF THE DRAWINGS

FIG. 1 is a longitudinal section of one embodiment of the present invention, a 2-stage cascade injection device in the charged state before injection;

FIG. 2 is a longitudinal cut of a device of FIG. 1 during the discharge phase;

FIG. 3 is a longitudinal cut of a device similar to FIG. 1, but utilizing a balloon in the form of a torus pressurized by nitrogen; and

FIG. 4 is a diagram showing the force generated as a function of the displacement (X) of the piston stem for different characteristics of springs B and C with respect to the simple entropic spring A.

### DETAILED DESCRIPTION

Referring to the Figures, including FIGS. 1 to 3, in one embodiment, the present invention comprises an injection device 1 for dispensing a liquid 6 under the skin 14 of a human patient or of an animal. The device 1 includes an ampoule 7 equipped with a piston 5 forming an integral part of the ampoule, a transmission element 4, a retention means 9 and force generating elements 10, 10a included between two or more free movable partitions 3a and a fixed partition 2a forming an integral part of the injector body, and a partition 3 joined to the movable piston 5. This (movable) partition 3 is connected to the stem of piston 4 in a fixed or adjustable manner. If adjustment of the stroke of the stem of piston 4 is necessary, then these two elements will be threaded. The free movable partitions 3a are those that slide on the stem of the piston 4. The free movable partitions 3, 3a can be provided with spacers (or stops), which permit, depending on their length L, modifying the form of the force/displacement curve and moving the point of inflection to a very precise value.

FIG. 1 shows the injector 1 in a charged condition just before injection. The injector 1 is composed of a cascade assembly or series of two entropic generators/springs 10 and 10a displaying different force/displacement characteristics. It is composed of the cellular reticulated polymer disk 10 for the element generating the low pressure, and of a solid polymer disk 10a for the primary pressure element serving to pierce the patient's skin. Each disk is pierced at its center by a hole permitting the stem of piston 4 to slide through. The diameter of the disks must be 20 to 30% smaller than the internal diameter of the injection body 2a or of the spacer 20 so that when these latter are flattened, the substance will be able to flow toward the outside without touching the outside wall, which would considerably modify its characteristics. The entropic spring does not change in volume while flattening. A free space 13 and 13a is arranged to avoid contact of the spring 10 and 10a with the housing 2a or the spacer 20.

Charging the injector is done by moving the movable partitions 3, 3a toward the fixed wall 2a by pulling on the stem of piston 4 by suitable mechanical means (e.g., lever arm, screw, etc.) or pneumatic means (e.g., an air actuator). The trigger 9 is secured on the stem of piston 4 in a manner to maintain the injector in the charged state, ready for use. By applying a pressure F on the trigger 9, it releases the stem of piston 4, which will push the piston 5 and eject the liquid 6 through the orifice 6b.

The single use ampoule 7 is composed of a glass or polycarbonate body 7 equipped at its end with a nozzle transforming the pressure energy into kinetic energy communicated to the microjet of liquid.

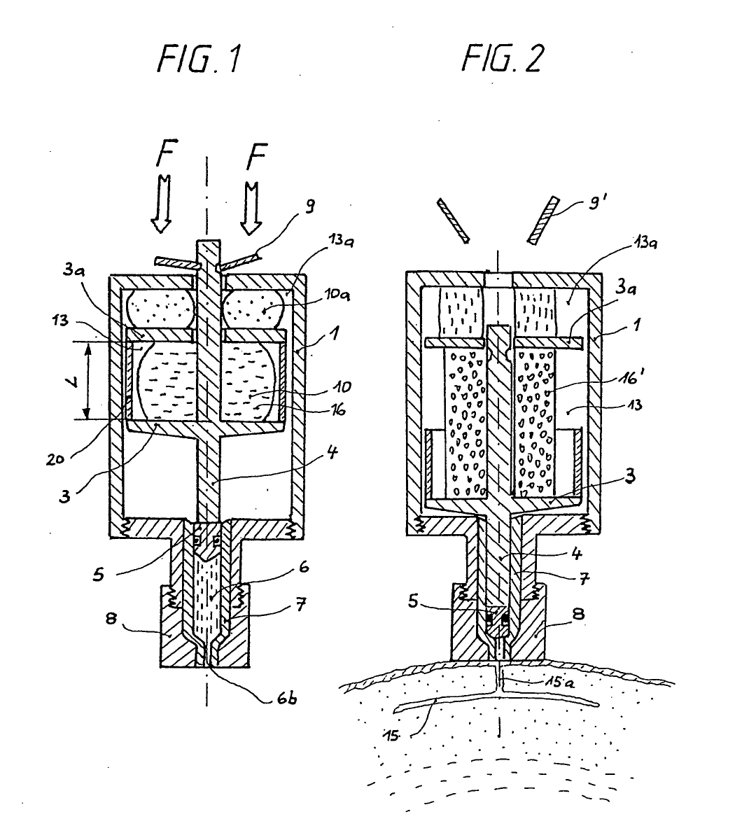

FIG. 2 shows the injector, after triggering, at the end of injection into the intradermal section 15. At the time of triggering the injector 1, the force generated by the spring 10a will create the relatively high pressure in the capsule 7 that is necessary to pierce the patient's skin (channel 15a). When relaxation of the spring 10a is complete, there then occurs shifting of the spring 10, which will generate a relatively lower pressure to permit the liquid to be transferred from the capsule to the zone 15 that is to be injected. In the case of a cellular spring like the one depicted in FIG. 1, the empty cells 16 are completely flattened, and at the time of relaxation as shown in FIG. 2, they retake their original forms 16.

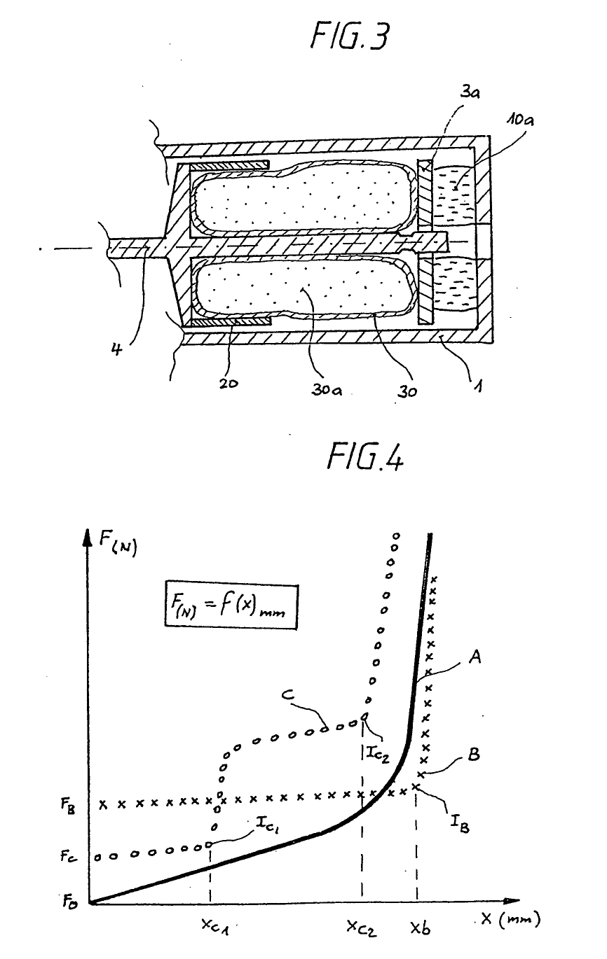

FIG. 3 shows a variant of FIG. 1 where the spring 10 is replaced by a balloon or rubber torus 30 pressurized by nitrogen. The nitrogen exhibits a low rate of diffusion through the rubber membrane, which permits maintaining the pressure over a long period of time without notable loss of pressure. This gas spring 30 will generate the secondary force necessary to transfer the liquid from the capsule 7 to zone 15. The primary force will be generated by the entropic spring 10a.

FIG. 4 shows a diagram of the force generated by the entropic spring as a function of the displacement of the stem of piston 4. The diameter of the piston 5 will determine the pressure in Mpa (Megapascal) that will be created inside the receiver 7 during injection. Curve A is the characteristic form of an entropic spring as described in applications PCT/IB02/02703 and PCT/IB02/04494 mentioned above. Curve B is the characteristic form generated by two springs assembled or arranged in cascade or series, and equipped with a spacer 20, as described in the present application. It is notable that the inflection point (I) associated with curve B is very distinct, which is not the case in curve A. An advantage is that the depth of injection is very precise and independent of the hardness of the skin. Another notable difference is the value of the force generated at the end of injection F2 due to a pre-stressing of the spring 10. The inflection point (I) at Xb can be moved on the abscissa (X) by modification of the length (L) of the spacer 20.

Curve C shows an assembly in cascade of three springs with different characteristics. This assembly comprises two free movable partitions 3a and 3b, and a movable partition 3 secured on the stem of the piston 4 equipped with a spacer 20. This arrangement permits generating (in series, sequentially or in steps) three different injection pressures during discharge of the injector.

FIGS. 1 and 2 show an injector whose trigger is located behind the entropic spring. It is also possible to produce the same injector with the trigger located between the ampoule and the entropic spring. Also, the possibilities for the number and arrangement of the kinds of springs, the number and arrangement of partitions, including the free movable partitions, forces generated, and the form of the reticulated polymer disks are not limited solely to those described in the patent, but encompass other suitable numbers, amounts, arrangements, substances and forms.

In the foregoing description, embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A hypodermic device for administration of a liquid product without a needle, said device comprising a reservoir containing the liquid product to be administered between a movable piston and an orifice, and at least two force generators arranged in cascade.

2. The device according to claim 1, wherein the piston is extended by a stem.

3. The device according to claim 2, wherein a first force generator is situated between the inside rear portion of the device and a movable partition, and a second force generator is situated between said movable partition and a partition joined to the piston.

4. The device according to claim 3, wherein the free movable partition slides on the stem of the piston.

5. The device according to claim 3, wherein additional force generators are situated between additional movable partitions.

6. The device according to claim 3, wherein the partition joined to the piston and the free movable partition have operatively associated spacers of a selected length.

7. The device according to claim 1, wherein the at least two force generators have different characteristics, the force generated thereby being a function of displacement of the piston.

8. The device according to claim 7, wherein the at least two force generators have different characteristics, the force generated thereby being a function of displacement of the piston thereby enabling an injection that is precise in amount as well as in depth.

9. The device according to claim 1, wherein the at least two force generators are at least one of the group comprising entropic springs, molecular springs, steel springs, magnetic springs or gas under pressure.

10. The device according to claim 1, wherein one of the at least two force generators is different from the other of the at least two force generators.

11. An injection device including a nozzle portion with an orifice, an ampoule containing a liquid to be injected, a piston, a retainer, and a force generator for generating at least two forces, said force generator comprising a partition moveable relative to the piston, a partition fixed to the piston, and a spring element between the moveable and fixed partitions, one of the least two forces sufficient to propel the liquid through the orifice in a jet of liquid having sufficient speed to pass through a patient's skin, and the other of the at least two forces sufficient to inject the liquid to a selected depth under the skin.

12. The injection device according to claim 11, wherein the other of the at least two forces is relatively lower that the one of the at least two forces.

13. An injection device for injecting a medicinal product, said device comprising a reservoir containing the medicinal product to be administered between a movable piston and an orifice, and at least two force generators arranged in series.

14. The device according to claim 13, wherein a first force generator is situated between an inside rear portion of the device and a movable partition, and a second force generator is situated between said movable partition and a partition joined to the piston.

15. A hypodermic injection device for intradermal or subcutaneous administration, without a needle, of a liquid product such as a medication or other pharmaceutical composition, said device comprising:
an ampoule containing said liquid to be injected;
at least two force generators assembled in cascade;
at least one movable partitions sliding on a stem of the piston;
a movable partition fixed to the piston;
spring elements between the movable and the fixed partitions;
a nozzle part having an orifice; and
retention means permitting maintaining, before utilization, the spring elements at a force sufficient to create in the ampoule a pressure to propel the liquid product to be injected, during utilization, through the orifice in a manner to create a jet of liquid having a sufficient speed to pass through the patient's skin and to inject the liquid to the correct depth.

16. The device according to claim 15, wherein the device is one of disposable after a single injection or suitable to perform multiple injections.
